(19) United States
(12) Patent Application Publication
(10) Pub. No.: US 2025/0127619 A1
(43) Pub. Date: Apr. 24, 2025

(54) CARDIAC PROSTHESIS DELIVERY DEVICE HAVING DEVICE POSITION FEEDBACK

(71) Applicant: Medtronic, Inc., Minneapolis, MN (US)

(72) Inventors: Ciaran M. McGuinness, Galway (IE); Stephen A. Montgomery, Galway (IE); Padraig P. Irwin, Dublin (IE); Ronan J. Benson, Sligo (IE); Alan J. Grincell, Leinster (IE); Mark M. Costello, Neale (IE)

(21) Appl. No.: 18/723,194

(22) PCT Filed: Feb. 6, 2023
(86) PCT No.: PCT/IB2023/051044

§ 371 Date: Jun. 21, 2024

Related U.S. Application Data
(60) Provisional application No. 63/310,372, filed on Feb. 15, 2022.

(51) Int. Cl. A61F 2/24

(52) U.S. Cl. CPC A61F 2/2436; A61F 2250/008

(57) ABSTRACT

Delivery devices for delivering a cardiac prosthesis to a target site are disclosed. Such devices can be used with a feedback system including haptic elements incorporated into the delivery device for providing tactile and/or audio feedback regarding the loading and/or deployment of the prosthesis. Certain disclosed delivery devices include a handle including an actuator, a sheath interconnected to the handle for selectively sheathing the prosthesis. The feedback system can be provided within the handle and configured to provide one or more feedback indications relating to the position of the sheath with respect to the prosthesis during loading and/or deployment of the prosthesis.

### FIELD OF THE DISCLOSURE

Aspects of present disclosure relate to delivery devices for cardiac prosthesis loading, delivery and implantation. The delivery devices include feedback systems including hepatic elements for providing at least one feedback indication communicating information regarding the loading and/or deployment of the prosthesis. In some embodiments, the prosthesis is a prosthetic heart valve.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are between the ventricles and the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of the valve prosthesis on the beating heart, intending to obviate the need for the use of classical sternotomy and cardiopulmonary bypass. In general terms, an expandable valve prosthesis is compressed about or within a catheter, inserted inside a body lumen of the patient, such as the femoral artery, and delivered to a desired location in the heart where the valve prosthesis is then deployed.

The disclosure presents improvements related to the above.

### SUMMARY

Aspects of present disclosure relate to delivery devices for cardiac prosthesis loading, delivery and implantation. Such delivery devices can include a sheath, a shaft at least partially positioned within the sheath and a handle connected to both the sheath and the shaft. Generally, the delivery devices provide a loaded delivery state in which the prosthesis is loaded and compressed over the shaft and further covered with the sheath. The delivery device can be manipulated to adjust the position of the sheath to permit the prosthesis to expand and ultimately release from the delivery device. In some embodiments, prior to full deployment of the prosthesis, the sheath can recapture the prosthesis for repositioning. Delivery devices of the disclosure include a feedback system for communicating information regarding the loading and/or deployment stage of the prosthesis. In examples of the disclosure, the feedback system is configured to provide a feedback indication communicated via tactile and/or audio feedback.

Aspects of the disclosure are beneficial in that they can be manufactured separately from a delivery device handle and can be separate from and spaced from other deployment and handle actuating mechanisms, which could potentially damage haptic elements of the feedback system during use. By providing the feedback system spaced from and not integrally formed with handle actuating mechanisms, a smoother device operation results, which improves the user experience. In addition, by being provided separately from other actuating mechanisms of the handle, haptic variables of the feedback system can also be altered or customized, as desired, without affecting other functions of the actuating mechanisms for creating a feedback system customized for a specific procedure or prosthesis.

In one aspect, the present disclosure provides a delivery device for delivering a stented prosthesis to a native heart valve. The delivery device includes a sheath and a shaft at least partially positioned coaxially within the sheath. The delivery device also includes a handle connected to the sheath and the shaft. The handle has a body having an inner surface and an actuator connected to the body. The actuator is configured to control linear movement of the sheath relative to the shaft. The handle additionally includes a feedback system having at least one ridge and a clip including a first flap extending from the sheath. The first flap is configured to contact and travel over the at least one ridge when the sheath moves longitudinally. The feedback system is configured to convey a first feedback indication when the flap contacts and travels past the at least one ridge. In various embodiments, the first feedback indication is one or more of audible feedback and tactile feedback that specifies a position of the sheath with respect to the shaft.

In another aspect, the disclosure provides methods including the step of providing a delivery device having a sheath as well as a shaft coaxially positioned at least partially within the sheath. A cardiac prosthesis is positioned on the shaft. The delivery device also includes a handle connected to the sheath and the shaft. The handle includes a body having an inner surface and an actuator connected to the body. The actuator is configured to control linear movement of the sheath. The handle further includes a feedback system having at least one ridge and a clip including a first flap extending from the sheath. The method includes moving the sheath in a first direction to a first position with respect to a distal end of the sheath with the actuator until the first flap contacts and travels past one or more of the at least one ridge to produce a first feedback indication. The first feedback indication can be one or more of audible feedback and tactile feedback.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1A is a perspective view of an example of a delivery device for delivering a cardiac prosthesis; the delivery device having a feedback system.

FIG. 1B is a schematic view of a handle assembly of the delivery device of FIG. 1A in a first position.

FIG. 1C is a schematic view of the handle assembly of FIGS. 1A-1B in a second position in which a sheath of the delivery device is distally advanced as compared to the first position of FIG. 1B.

FIG. 2 is a cross-sectional view of the feedback system of FIGS. 1A-1C having a cap, a core clip and a plurality of ridges or ribs.

FIG. 3 is a perspective view of the cap of FIG. 2.

FIG. 4 is a perspective view of the core clip of FIG. 2.

FIGS. 5A-5C are schematic illustrations of the feedback system of FIGS. 1A-2 as the cap moves with respect to the core clip to provide a feedback indication.

FIG. 6 is a perspective view of one example of a haptic rib insert having three sections, each section including a plurality of ridges or ribs and each section configured to provide a different feedback indication.

FIG. 7 is a perspective view of another example of a haptic rib insert having two sections, each section including a plurality of ridges or ribs and each section configured to provide a different feedback indication.

FIG. 8 is a perspective view of yet another example of a haptic rib insert having three sections, each section including a plurality of ridges or ribs and each section configured to provide a different feedback indication.

FIG. 9 is a perspective view of an alternate clip that can be used with embodiments of the disclosure.

FIG. 10 is a perspective view of another clip that can be used with embodiments of the disclosure.

FIG. 11 is a perspective view of yet another clip that can be used with embodiments of the disclosure.

FIG. 12 is a perspective view of an alternate feedback system that can be incorporated into a delivery device, such as that of FIGS. 1A-1C.

FIGS. 13A-13D are schematic illustrations of the feedback system of FIG. 12 illustrating how the feedback system is configured to provide a feedback indication as the clip moves in one direction but not in the opposite direction.

### DETAILED DESCRIPTION

Specific embodiments of the present disclosure are now described with reference to the figures, wherein like reference numbers indicate identical or functionally similar elements.

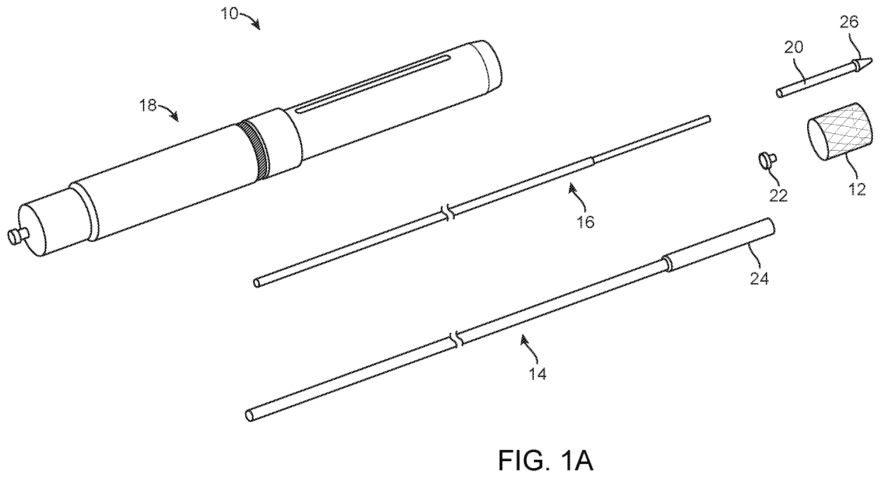

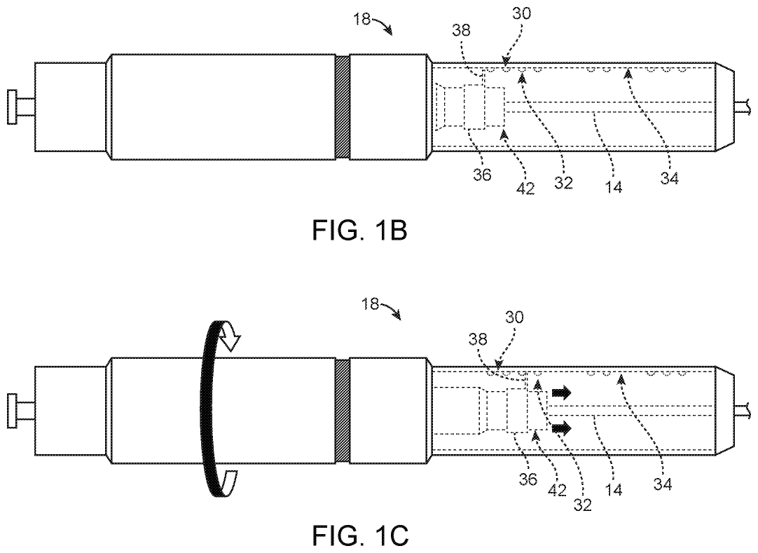

As described below, aspects of the present disclosure relate to feedback systems having haptic elements for incorporating into delivery devices for delivering and deploying a cardiac prosthesis at a target site, such as a native heart valve. By way of background, general components of one non-limiting example of a delivery device 10 with which some embodiments of the present disclosure are useful are illustrated FIGS. 1A-1C. The delivery device 10 is arranged and configured for percutaneously delivering a cardiac prosthesis 12, such as a stented prosthetic heart valve, stent or the like. Generally, the delivery device 10 includes a sheath 14, a shaft 16 and a handle 18. The prosthesis 12 is loaded onto a distal portion 20 of the shaft 16, optionally with a prosthesis retainer 22 configured to engage and maintain the prosthesis 12 on the shaft 16 while compressed within the sheath 14. The sheath 14 and/or shaft 16 may be a singular, integrally formed component or can alternatively include multiple interconnected components. In some examples, the sheath 14 includes a capsule 24 in which the loaded prosthesis 12 is compressed in a delivery state while positioned during delivery. In some examples, the distal portion 20 of the shaft 16 is a separate component that includes a tip 26, which can be conically shaped or otherwise adapted to promote atraumatic contact with bodily tissue.

Once loaded, compressed and covered by the sheath 14, the prosthesis 12 is delivered to the target site, such as a native heart valve. When the prosthesis 12 is at the target site, the sheath 14 and capsule 24 are proximally withdrawn with respect to the distal portion 20 and prosthesis 12 loaded thereto to permit the prosthesis 12 to expand to an expanded arrangement, partially releasing and ultimately fully deploying the prosthesis 12 from the shaft 16/retainer 22. The prosthesis 12 can be expanded using any known technique such as with an expandable balloon or naturally if the prosthesis 12 is made of a shape memory material configured to be biased to the expanded arrangement. Should recapture of the prosthesis 12 be desired for either repositioning of the prosthesis or bailout of the procedure, the capsule 24 can be distally advanced prior to full deployment of the prosthesis. Distal and proximal movement of the sheath 14 and capsule 24 relative to the stented prosthesis 12 can be actuated by the handle 18. The handle 18 can take many configurations for both supporting the sheath 14 and shaft 16 as well as controlling movement of each of these components. In one example, the handle 18 can include an actuator 28 being a rotatable portion such that rotational movement of the actuator 28 about a central longitudinal axis of the handle 18 translates to movement of the sheath 14 to correspondingly move the capsule 24 to either sheathe or unsheathe the prosthesis 12.

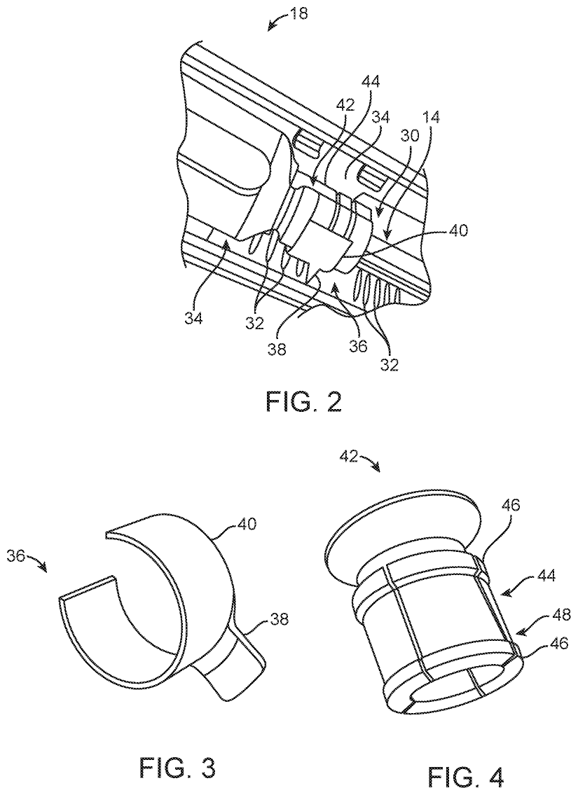

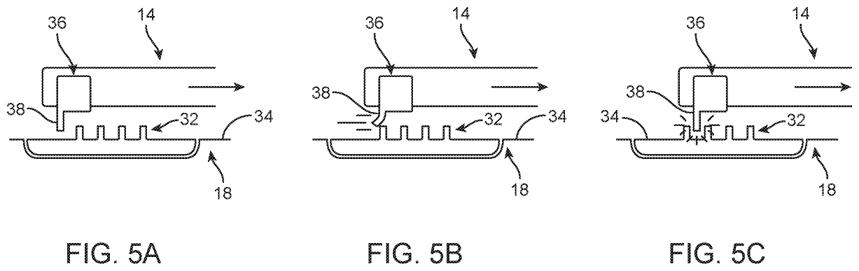

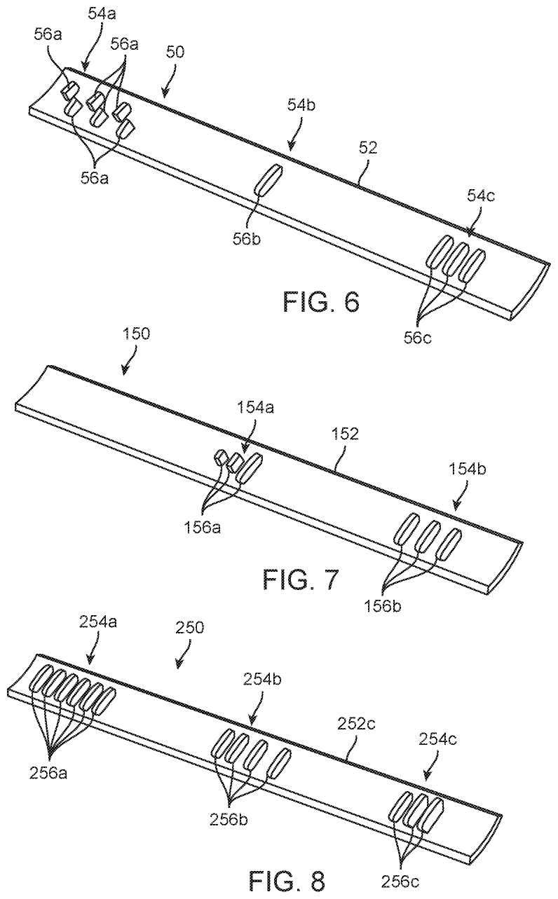

Various embodiments of the disclosure include a haptic feedback system 30 incorporated into the handle 18 to provide a clinician with feedback relating to the loading and/or deployment of the prosthesis 12. Haptic feedback systems 30 of the disclosure can include a plurality of ridges or ribs 32 (generally referenced) provided on an inner surface 34 of the handle 18 and a cap 36 having a flap 38 configured to interact with the ribs 32 as the cap 36 moves across the ribs 32. The flap 38 is made of a flexible material so that the flap can bend and pass over the ribs 32 as is generally shown in FIGS. 2 and 5A-5C. In some embodiments, the ribs 32 can be provided directly on the inner surface 34 and in other examples, the ribs 32 can be provided on a strip of material (see also, FIGS. 6-8) that is applied to the inner surface 34 of the handle 18.

One example of the cap 36 is best shown in FIGS. 2-3. In this example, the cap 36 includes a base 40 maintaining the flap 38. The base 40 can be semi-circular to snap onto the shaft 16. In other examples, the base 40 can otherwise be secured to a core clip 42, which can optionally be considered part of the sheath 14. In the example of FIG. 4, the core clip 42 can include a generally cylindrical portion 44 having two raised guides 46 between a channel 48 sized to receive and maintain a position of the base 40 with respect to the distal portion 20 of the sheath 14. Any other ways of securing the flap 38 to the sheath 14 are also considered within the scope of the present disclosure.

Ribs or ridges of the disclosure be configured in a variety of ways to provide a feedback indication conveying information to a clinician regarding multitude of sheath/capsule stages, as desired. For example, the positioning of the ribs can provide a feedback indication to inform a user when to change a pace of the prosthesis deployment or when to stop deployment or proximal retraction of the sheath. Further, during loading procedures, the ribs can be provided and configured to provide a feedback indication to inform a user when they are entering the tip overdrive region during loading of the prosthesis within the sheath. The aforementioned stages of loading and deployment of the prosthesis are considered to be non-limiting examples of the disclosure.

Various embodiments are configured to provide multiple feedback indications, which differ from each other in one or more of tactical feel or audible sound to clearly distinguish the feedback indication. To achieve differing tactical feel or audible sounds, the plurality of ribs can be differently configured. In the example of FIG. 6, a haptic rib insert 50 can be provided for application to the inner surface 34 of the handle 18 (see also FIGS. 1A-2). The haptic rib insert 50 includes a strip of material 52 having three sections 54a, 54b, 54c, each having a plurality of ribs or ridges 56a, 56b, 56c (ribs being generally referenced). The sections 54a, 54b, 54c can alternatively be provided directly on the inner surface 34 of the handle 18. Each section 54a, 54b, 54c can designate a state of prosthesis 12 loading or deployment based on the position of the sheath 14 with respect to the distal portion 20 of the shaft 16, for example. In this embodiment, the first section 54a includes a plurality of ribs 56a having a uniform longitudinal spacing, uniform horizontal spacing and uniform height to provide a first feedback indication as a flap (e.g., flap 38) travels across the three ribs 56a. The second section 54b includes a singular rib 56b to provide a second feedback indication including tactical and audio feedback as the flap travels across the rib 56b that differs from the first feedback indication. In a third section 54c, a plurality of ribs 56c are provided that differ in number from the number of ribs in the first and second sections 54a, 54b. In addition, the plurality of ribs 56c of the third section 54c are staggered in horizontal placement, which can result in a third feedback indication including tactical and audial feedback differing from the first and second feedback indications as the flap travels over the plurality of ribs 56c.

In a haptic rib insert 150 example of FIG. 7, the insert 150 includes a strip of material 152 having two sections 154a, 154b, each section including a plurality of ribs or ridges 156a, 156b. The plurality of ribs 156a of the first section 154a have uniform height and length as well as uniform longitudinal and horizontal spacing. The plurality of ribs 156b of the second section 154b having uniform longitudinal spacing but differing lengths and heights. The properties of the plurality of ribs 156a of the first section differ 154a from the properties of the plurality of ribs 156b of the second section 154b so that the tactile and/or audio feedback provided as a flap (e.g., flap 38) traverses the respective sections 154a, 154b differs from the first section 154a to the second section 154b.

In a haptic rib insert 250 example shown in FIG. 8, the insert 250 includes a strip of material 252 having three sections 254a, 254b, 254c, each section including a plurality of ribs 256a, 256b, 256c. The plurality of ribs or ridges 256a, 256b of each section 254a, 254b, 254c having uniform height and width. The plurality of ribs 256a, 256b, 256c at each section 254a, 254b, 254c having a differing number of ribs as well as differing longitudinal spacing so that the tactile and/or audio feedback provided as the flap traverses the respective sections 254a, 254b, 254c differs from the first section 254a to the second section 254b and also the third section 254c. In the example of the third section 254c, the ribs 256c stepped or increasing in height to result in an increasingly loud noise as the flap traverses the section 254c. It will be understood that the section and rib examples provided in here are merely illustrative and that the concepts of varying ribs and sections is not limited those shown.

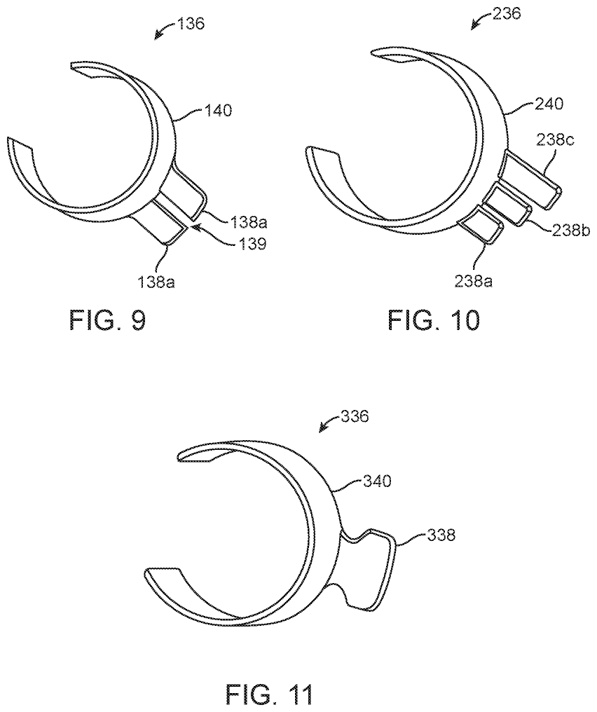

Referring now in addition to FIGS. 9-11, clips of the present disclosure can take a variety of configurations. In the example of FIG. 9, a clip 136 can include a body 140 and two flaps 138a, 138b separated by a gap 139, each flap 138a, 138b having a uniform length. Depending on the horizontal spacing of the plurality of the ribs, one flap may contact a rib or ridge whereas the other may not, which would result in a variance in the resultant sound or feel as the clip traverses the section. In the example of FIG. 10, a clip 236 can include a body 240 and a plurality of flaps 238a, 238b, 238c, each flap 238a, 238b, 238c having a different length. In combination with ribs having differing lengths, this configuration will allow certain flaps to pass over ribs in one section but not another section (i.e. produce one or more of an audible sound or tactile event when traversing one section or one rib but not another). In the example of FIG. 11, a clip 336 can include a body 340 and at least one flap 338 having a width that is non-uniform along its length.

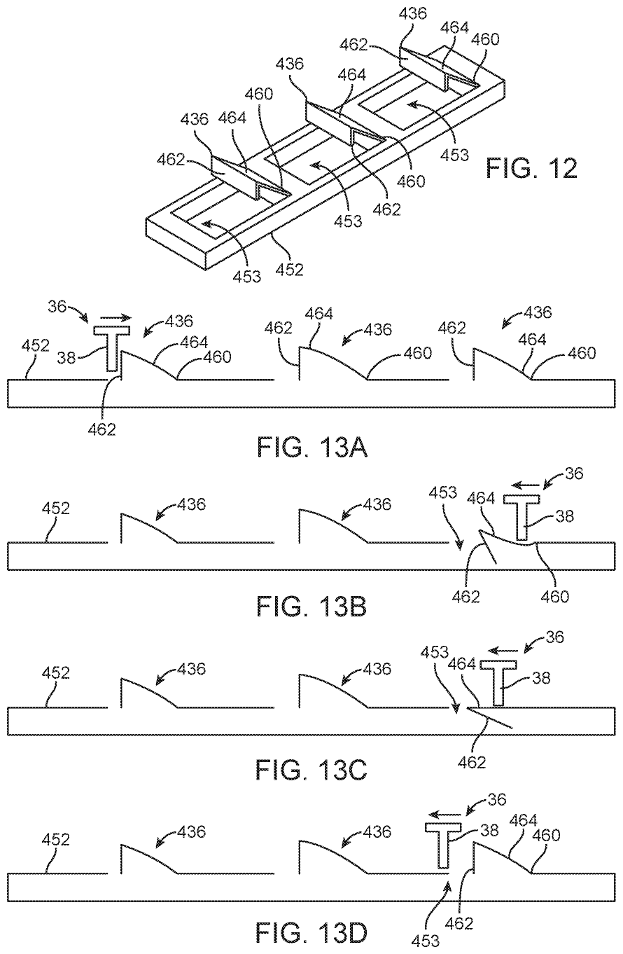

Referring now in addition to FIGS. 12-13D, in various embodiments one or more of sections of the disclosure can be configured such that a plurality of ribs or ridges 436 provide feedback when the sheath and flap 38 advances in one direction (i.e. proximal or distal directions) but not in the opposite direction. In such embodiments, it may be determined that feedback, such as sound or vibration, resulting from movement in one direction is not useful and is therefore, desirably omitted. In one example, each rib 436 can be connected to the inner surface of the handle (either via strip, such as strip 452 or otherwise) at a hinged portion 460. Opposite the hinged portion 460, the rib 436 can include a contact portion 462 that is angled with respect to a ramped portion 464 interconnecting the hinged portion 460 and the contact portion 462. The inner surface or strip 452 can include one or more pockets 453 sized to receive one respective rib. As shown in FIG. 13A, as the flap 38 advances in one direction (e.g., distally), the flap 38 contacts and advances over the contact portion 462 to produce a feedback indication (i.e. sound or tactile vibration). As shown in FIGS. 13B-13D, as the flap 38 moves in the opposite direction, the flap 38 will travel over the hinged portion 460 and along the ramped portion 464, pushing the flap 38 at least partially into the respective pocket 453. When the ramped portion 464 is adjacent the contact portion 462, the contact portion 462 will be within the pocket 453, allowing the flap 38 to smoothly travel past the respective rib 436 without generating a feedback indication. Any of the embodiments disclosed herein can include one or more sections or ribs configured in this way, to provide the desired feedback indication.

Various methods of the disclosure can be summarized as follows. In one example, a method includes the step of providing a delivery device having a sheath as well as a shaft coaxially positioned at least partially within the sheath. A cardiac prosthesis is positioned on the shaft. The delivery device also includes a handle connected to the sheath and the shaft. The handle includes a body having an inner surface and an actuator connected to the body. The actuator is configured to control linear movement of the sheath. The handle further includes a feedback system having at least one ridge and a clip including a first flap extending from the shaft. The method includes moving the shaft in a first direction to a first position with respect to a distal end of the sheath with the actuator until the first flap contacts and travels past one or more of the at least one ridge to produce a first feedback indication. The first feedback indication can be one or more of audible feedback and tactile feedback.

In some methods, the first feedback indication conveys information indicating a deployment stage of the cardiac prosthesis. In other examples, the first feedback indication conveys information indicating compression of the shaft during a step of sheathing the cardiac prosthesis with the sheath. In some embodiments, at least one ridge is a plurality of ridges arranged in a first section of the body and a plurality of ridges arranged in a second section of the body, the first position being at the first section; wherein the method further includes using the actuator to move the sheath to a second position with respect to the shaft until the first flap contacts and travels over one or more of the plurality of ridges of the second section to produce a second feedback indication. Optionally, the plurality of ridges at the first section differs in construction as compared to the plurality of ridges at the second section. In some methods, the first feedback indication and the second feedback indication are audible and the first feedback indication differs in sound from the second feedback indication. In some examples, an audible volume of the first feedback indication differs from an audible volume of the second feedback indication. Optionally, a number of audible clicks of the first feedback indication differs from a number of audible clicks of the second feedback indication. In some examples, the at least one ribs are static with respect to the body during the step of moving the catheter assembly to produce the first feedback indication. In one example, the first feedback indication can include two audible clicks. Some methods can further include moving the sheath in a second direction, opposite the first direction; wherein, while the sheath is moving in the second direction, the feedback system does not produce an audible or tactile indication as the flap contacts and travels over one or more of the at least one ridge.

As referred to herein, cardiac prostheses and stented prosthetic heart valves useful with the various devices and methods of the present disclosure may assume a wide variety of configurations, such as a bioprosthetic heart valve having tissue leaflets or a synthetic heart valve having polymeric, metallic or tissue-engineered leaflets, and can be specifically configured for replacing valves of the human heart. The stented prosthetic heart valves and other stented prostheses of the present disclosure may be self-expandable, balloon expandable and/or mechanically expandable or combinations thereof. In general terms, the stented prostheses of the present disclosure include a stent or stent frame having an internal lumen maintaining a valve structure (tissue or synthetic), with the stent frame having a normal, expanded condition or arrangement and collapsible to a compressed condition or arrangement for loading within the delivery device. For example, the stents or stent frames are support structures that comprise a number of struts or wire segments arranged relative to each other to provide a desired compressibility and strength to the stented prosthesis. The struts or wire segments are arranged such that they are capable of self-transitioning from, or being forced from, a compressed or collapsed arrangement to a normal, radially expanded arrangement. The struts or wire segments can be formed from a shape memory material, such as a nickel titanium alloy (e.g., nitinol). The stent frame can be laser-cut from a single piece of material, or can be assembled from a number of discrete components.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A delivery device for delivering a cardiac prosthesis to a target site, the delivery device comprising:
a sheath;
a shaft at least partially positioned coaxially within the sheath;
a handle connected to the sheath and the shaft, the handle including:
a body having an inner surface,
an actuator connected to the body and configured to control linear movement of the sheath relative to the shaft, the handle further including a feedback system having:
at least one ridge, and
a clip including a first flap extending from the sheath; the first flap being configured to contact and travel over the at least one ridge when the sheath moves longitudinally;
wherein the feedback system is configured to convey a first feedback indication when the first flap contacts and travels past the at least one ridge;
further wherein the first feedback indication is selected from the group consisting of audible feedback, tactile feedback and a combination thereof that specifies a position of the sheath with respect to the shaft.

2. The delivery device of claim 1, wherein the feedback system includes a plurality longitudinally spaced ridges.

3. The delivery device of claim 2, wherein the plurality longitudinally spaced ridges are positioned in a first section on the body including a first set of longitudinally spaced ridges and a second section on the body including a second set of longitudinally spaced ridges; wherein a configuration of the first set differs from a configuration of the second set.

4. The delivery device of claim 2, wherein at least three of the plurality of ridges are varied in one or more of longitudinal or vertical spacing with respect to adjacent ridges of the plurality of ridges.

5. The delivery device of claim 2, wherein at least two of the plurality of ridges have different widths.

6. The delivery device of claim 2, wherein at least two of the plurality of ridges have different heights.

7. The delivery device of claim 1, wherein the clip includes a second flap.

8. The delivery device of claim 7, wherein the second flap has a length that is different than a length of the first flap.

9. The delivery device of claim 1, wherein the at least one ridge is configured such that the first flap provides the first feedback indication when the first flap travels over the at least one ridge in a first direction but not when the first flap travels over the ridge in a second direction.

10. A method comprising:
providing a delivery device including:
a sheath;
a shaft coaxially positioned at least partially within the sheath and having a distal portion;
a cardiac prosthesis positioned on the distal portion;
a handle connected to the sheath and the shaft, the handle including:
a body having an inner surface,
an actuator connected to the body and configured to control linear movement of the sheath, the handle further including a feedback system having:
at least one ridge, and
a clip including a first flap extending from the sheath; and
moving the sheath in a first direction to a first position with respect to a distal end of the shaft with the actuator until the first flap contacts and travels past one or more of the at least one ridge to produce a first feedback indication selected from the group consisting of audible feedback, tactile feedback and a combination thereof.

11. The method of claim 10, wherein the first feedback indication conveys information indicating a deployment stage of the cardiac prosthesis.

12. The method of claim 10, wherein the first feedback indication conveys information indicating compression of the cardiac prosthesis during a step of sheathing the cardiac prosthesis with the sheath.

13. The method of claim 10, wherein the at least one ridge is a plurality of ridges arranged in a first section of the body and a plurality of ridges arranged in a second section of the body, the first position being at the first section; wherein the method further includes using the actuator to move the sheath to a second position with respect to the shaft until the first flap contacts and travels over one or more of the plurality of ridges of the second section to produce a second feedback indication.

14. The method of claim 13, wherein the plurality of ridges at the first section differs in construction as compared to the plurality of ridges at the second section.

15. The method of claim 14, wherein the first feedback indication and the second feedback indication are audible and the first feedback indication differs in sound from the second feedback indication.

16. The method of claim 14, wherein an audible volume of the first feedback indication differs from an audible volume of the second feedback indication.

17. The method of claim 14, wherein a number of audible clicks of the first feedback indication differs from a number of audible clicks of the second feedback indication.

18. The method of claim 10, wherein the at least one ridge is static with respect to the body during the step of moving the sheath in the first direction to produce the first feedback indication.

19. The method of claim 10, wherein the first feedback indication includes two audible clicks.

20. The method of claim 10, further comprising moving the sheath in a second direction, opposite the first direction; wherein, while the sheath is moving in the second direction, the feedback system does not produce an audible or tactile indication as the flap contacts and travels over one or more of the at least one ridge.
